(19) United States
(12) Patent Application Publication
(10) Pub. No.: US 2012/0302857 A1
(43) Pub. Date: Nov. 29, 2012

(54) BRAIN SIGNAL MEASUREMENT SYSTEM AND MEASUREMENT SYSTEM

(75) Inventors: Toshitaka Yamakawa, Fukuoka (JP); Takeshi Yamakawa, Fukuoka (JP)

(73) Assignee: KYUSHU INSTITUTE OF TECHNOLOGY, Kitakyushu-shi, Fukuoka (JP)

(21) Appl. No.: 13/575,023

(22) PCT Filed: Jan. 24, 2011
(86) PCT No.: PCT/JP2011/051237

§ 371 Date: Jul. 24, 2012

(30) Foreign Application Priority Data
Jan. 25, 2010 (JP) 2010-013482

(51) Int. Cl. A61B 5/0478; A61B 5/0476

(52) U.S. Cl. 600/378; 600/544

(57) ABSTRACT

Proposed are a brain signal measurement system and the like, capable of carrying out in vivo placement of measurement units for measuring brain signals with a minimally invasive operation, as well as of easily adjusting the position of each measurement unit. The brain signal measurement device includes sensors to be provided in a space between a dura mater and an arachnoid mater, and a holding unit for holding the sensors. The sensors are connected by a shape-memory unit. The sensors are inserted through a hole penetrating through the scalp and so on, into a space between the dura mater and the arachnoid mater. The diameter of the hole can be equal to or smaller than 1 cm. By electrical heating, the shape-memory unit changes its shape to change positions of the sensors, and thus subdural electrodes can be placed with a minimally invasive operation.

### TECHNICAL FIELD

The present invention relates to brain signal measurement systems and measurement systems, and in particular, to a brain signal measurement system and the like, including a brain signal measurement device having a plurality of measurement units provided in a space between a dura mater and an arachnoid mater and each configured to measure a brain signal, and a holding unit configured to hold the plurality of measurement units.

### BACKGROUND ART

Measurement of brain signals is used in various medical treatments such as measurement of brain signals in an epileptic seizure, for example. In general, measurement of brain signals is performed either by placing electrodes on a surface of the head of a patient, or by intracranially placing intracranial electrodes to measure brain signals. Examples of commonly used intracranial electrodes for intracranially measuring brain signals include subdural electrodes placed on a brain surface, and deep brain electrodes inserted in deep brain parenchyma (see Patent Document 1).

Generally, placement of subdural electrodes is carried out through a craniotomy procedure of a site at which the electrodes are placed. Patent Document 2 describes percutaneously inserting a device into a subarachnoid space from a certain ingression site. Patent Document 3 describes causing an arm to hold a neuromodulation assembly (NMA) in a flexed manner, inserting the NMA to a portion near corpus callosum via bushing, and spreading the NMA from a distal end opening of the bushing to place the NMA at a target position. Patent Document 4 describes, in order to measure physiological signals of the heart, inserting a catheter, spreading a ring in the heart, and arranging electrodes provided for the ring.

### PRIOR ART DOCUMENTS

### Patent Documents

### Patent Document 1: JP 2009-45368 A

### Patent Document 2: JP 2004-534590 W

### Patent Document 3: US 2005/0,288,760 A

### Patent Document 4: JP 2001-502189 A

### SUMMARY OF THE INVENTION

### Problems to be Solved by the Invention

However, when placing subdural electrodes by a conventional craniotomy procedure, it is highly invasive as requiring a craniotomy procedure to be performed to a large area. In addition, because general anesthesia is necessary not only in placement but also in removal, it is necessary to perform a craniotomy procedure to a large area at least two times within a predetermined period. This places a tremendous burden on the patient. Additionally, as general anesthesia is necessary in order to perform such a craniotomy procedure to a large area, it is necessary to secure a large number of doctors including anesthesists. Therefore, an operation for placing subdural electrodes has been difficult to be conducted because of an influence to the patient in terms of high invasiveness, and of a difficulty in adjustment of schedules of the doctors in advance.

Further, the technique described in Patent Document 2 basically relates to one dimensional back-and-forth insertion using a guide wire and the like. It is necessary to arrange a plurality of electrodes three dimensionally, and therefore it is difficult to place the plurality of electrodes respectively at desired positions by such one dimensional adjustment.

Moreover, the technique described in Patent Document 3 relates to in vivo placement of an NMA by inserting the NMA into the body while being sandwiched and deformed by an arm made of a rigid material, and releasing the pressure of the arm near a site of arrangement to restore the shape of the NMA. As the arm made of a rigid material is inserted near the arrangement site, this may possibly damage the brain. In addition, it is difficult to adjust the position of the NMA after the NMA is separated from the arm and arranged within the body. According to the technique described in Patent Document 4, the ring is made of an elastic material. The ring forms a circular shape within the heart due to the elasticity. Therefore, Patent Document 4 poses the same problem as the technique described in Patent Document 3.

Difficulties described above in placement of measurement units for in vivo measurement of signals are not limited to measurement of brain signals, and also apply to measurement of signals from a self-contained pacemaker, for example.

Thus, an object of the present invention is to propose a brain signal measurement system and the like, capable of carrying out in vivo placement of a plurality of measurement units for measuring signals with a minimally invasive operation, as well as of easily adjusting a position to place each of the measurement units.

### Solutions to the Problems

The first aspect of the present invention is a brain signal measurement system, comprising a brain signal measurement device including a plurality of measurement units provided in a space between a dura mater and an arachnoid mater, and each configured to measure a brain signal, and a holding unit configured to hold the plurality of measurement units, wherein the holding unit includes a shape-memory unit having a shape-memory property exerted by predetermined stimulation, and configured to change positions of the plurality of measurement units by a shape change of the shape-memory unit based on the shape-memory property, a stimulus introduction unit configured to supply stimulation to the shape-memory unit from exterior and a stimulation blocking unit covering the shape-memory unit, and configured to block an influence given to exterior by a predetermined phenomenon other than the shape change of the shape-memory unit by supplying the predetermined stimulation to the shape-memory unit from exterior, the holding unit in a non-stimulated state is more flexible than the dura mater and the arachnoid mater, and the shape-memory unit changes the shape thereof into a shape previously memorized through a shape-memory treatment and positions the plurality of measurement units, the change of the shape being based on the shape-memory property, the change of the shape being caused by providing the holding unit between the dura mater and the arachnoid mater and by supplying the predetermined stimulation using a stimulation device via the stimulus introduction unit.

The second aspect of the present invention is the brain signal measurement system of the first aspect, wherein the shape-memory unit is configured by a shape-memory alloy, and changes the shape thereof based on the shape-memory property by being heated up to or above a predetermined temperature, the stimulation supplied by the stimulation device is electrical heating, and the stimulation blocking unit is a thermal and electrical insulator.

The third aspect of the present invention is the brain signal measurement system of the first or the second aspect, wherein each of the measurement units is configured by a transmission wire having one end configuring an electrode, the wire being electrically connected to a measurement device and capable of transmitting a measurement result obtained at the electrode, a portion of the transmission wire other than the electrode is coated, and at least a part of the portion of the transmission wire other than the electrode is covered by the stimulation blocking unit in addition to the coating so as to allow transmission of the measurement result obtained at the electrode while blocking an influence given to and from exterior, and the electrode is configured by the transmission wire with its coating separated and is wound around the holding unit.

The fourth aspect of the present invention is the brain signal measurement system of any of the first through the third aspect, wherein the shape-memory unit is shape-memory treated to memorize a polygonal shape having apices of a number of or greater than a number of the plurality of measurement units, the measurement units are respectively held at the apices of the polygonal shape of the holding unit.

The fifth aspect of the present invention is the brain signal measurement system of the fourth aspect, wherein an imaging device including an imaging unit capable of exogenously taking an image of the shape-memory unit and a position estimation unit configured to perform image processing to information of the image taken by the imaging unit estimate a position of a detected one of the measurement units based on a detected position and estimate a position of an un-detected one of the measurement units to be a position of an apex estimated based on information of sides of the polygonal shape of the shape-memory unit.

The sixth aspect of the present invention is an in vivo measurement device, comprising a plurality of measurement units provided in a predetermined in vivo space, and each configured to measure a signal and a holding unit configured to hold the plurality of measurement units, wherein the holding unit includes a shape-memory unit having a shape-memory property exerted by predetermined stimulation, and configured to change positions of the plurality of measurement units by a shape change of the shape-memory unit based on the shape-memory property, a stimulus introduction unit configured to supply stimulation to the shape-memory unit from exterior and a stimulation blocking unit covering the shape-memory unit, and configured to block an influence given to exterior by a predetermined phenomenon other than the shape change of the shape-memory unit by supplying the predetermined stimulation to the shape-memory unit from exterior, the holding unit in a non-stimulated state is more flexible than an outer wall of the predetermined in vivo space, the shape-memory unit changes the shape thereof into a shape previously memorized through a shape-memory treatment and positions the plurality of measurement units, the change of the shape being based on the shape-memory property, the change of the shape being caused by providing the holding unit in the predetermined in vivo space and by supplying the predetermined stimulation using a stimulation device via the stimulus introduction unit.

The seventh aspect of the present invention is a brain signal measurement position control method for controlling a holding unit of a brain signal measurement device having a plurality of measurement units provided in a space between a dura mater and an arachnoid mater and each configured to measure a brain signal and a holding unit configured to hold the plurality of measurement units, wherein the holding unit includes a shape-memory unit having a shape-memory property exerted by predetermined stimulation, and configured to change positions of the plurality of measurement units based on the shape-memory property, a stimulus introduction unit configured to supply stimulation to the shape-memory unit from exterior; and a stimulation blocking unit covering the shape-memory unit, and configured to block an influence given to exterior by a predetermined phenomenon other than a shape change of the shape-memory unit by supplying stimulation to the shape-memory unit from exterior, and the holding unit in a non-stimulated state is more flexible than the dura mater and the arachnoid mater, the method comprising the step of changing the shape of the shape-memory unit of the holding unit into a shape previously memorized through a shape-memory treatment and positioning the plurality of measurement units, the change of the shape being based on the shape-memory property, the shape change being caused by providing the holding unit between the dura mater and the arachnoid mater and by supplying the predetermined stimulation using a stimulation device via the stimulus introduction unit.

It should be noted that the present invention is considered to be used not only in insertion, but also in removal. Specifically, it is possible to provide a state in which stimulation is not applied from the stimulation device to the shape-memory unit, and then the holding unit is removed from the space between the dura mater and the arachnoid mater. With this, in removal, it is possible to provide the same condition as that in insertion into the body, maintaining flexibility without exhibiting the shape-memory property. Thus, the brain signal measurement device can be removed with a minimally invasive operation.

Further, the brain signal measurement system and the like according to the present invention may further include the introduction tubes having a pair of tubes provided in parallel with one end of each of the pair of the tubes to be inserted into the head through. The holding unit may be configured as a line, passing through one of the introduction tubes from one end which is not to be inserted into the head and exiting from the other end which is to be inserted into the head. Then, the line is further inserted into the other of the introduction tubes from one end which is to be inserted into the head and exiting from the other end which is not to be inserted into the head. Arrangement using such introduction tubes further facilitates the placement of the shape-memory unit even when the shape-memory unit is electrically heated.

Moreover, the brain signal measurement system and the like according to the present invention may further include the imaging device having the imaging unit capable of exogenously taking an image of the shape-memory unit, and a display configured to display information taken by the imaging unit.

Furthermore, the shape-memory unit may be made of a shape-memory alloy, for example. Examples of a material of the shape-memory alloy include a titanium-nickel alloy. This alloy has been proved to be biocompatible, and variously utilized in medical applications including applications for biological implantation. Other examples may include an iron-based shape-memory alloy. Further, examples of a material of the measurement units include platinum (platinum) and a platinum iridium alloy. These alloys have also been proved to be biocompatible, and variously utilized in medical applications including applications for electrodes for catheters and pacemakers. Further, examples of a material of the stimulation blocking unit (insulation coating) include a fluorine resin as PTFE. Likewise, there is a fluorine resin whose biocompatibility has been proved.

Further, the present invention may be considered as a program for causing a computer to operate as the position estimation unit according to the fifth aspect of the present invention, and a computer-readable recording medium having the program recorded therein. In addition, the brain signal measurement system and the like, according to the present invention, may further include a display for displaying an estimated position may be provided. Additionally, an input unit through which a user inputs positional information of each measurement unit and a comparison unit configured to compare the inputted positional information of each measurement unit with estimated positional information may be further included, and the display may display the comparison results.

### Effects of the Invention

According to the present invention, the holding unit holds the plurality of measurement units, is flexible when being inserted into the body while the shape-memory unit connecting the measurement units is not supplied with stimulation (for example, flexible in an environment in temperature from room temperature to body temperature in a case in which the shape-memory property is exhibited by a temperature change), and arranges the plurality of measurement units by being inserted in this state into a predetermined in vivo space between the dura mater and the arachnoid mater, for example, applying predetermined stimulation to the shape-memory unit in the body, and changing the shape of the shape-memory unit into the shape previously memorized through the shape-memory treatment. The hole for insertion can be realized as a hole having a diameter equal to or smaller than 1 cm, for example. Here, the holding unit being more flexible than the dura mater and the arachnoid mater for example means hardness of a degree in which, as there is a space between the dura mater and the arachnoid mater, it is possible to continue the insertion into the space between the dura mater and the arachnoid mater even if a pressure is applied from the holding unit to the dura mater and the arachnoid mater as the holding unit is inserted.

Further, according to the present invention, there are provided the stimulus introduction unit and the stimulation blocking unit, and the holding unit is flexible when being inserted into the body as stimulation is not being supplied, and stimulation is applied to the shape-memory unit within the body. Therefore, unlike the technique described in Patent Document 3, a portion to be inserted into the body with no stimulation is more flexible than the dura mater and the arachnoid mater, for example, and less likely to damage the dura mater and the arachnoid mater in insertion.

Moreover, even after stimulation is once applied, the position of the holding unit in the body can be easily adjusted by restoring the flexible state by restoring a state in which no stimulation is applied, adjusting the position of the holding unit, and again supplying stimulation. Therefore, the measurement units can be placed with a minimally invasive operation, and it is possible to easily adjust the positions of the plurality of measurement units in the body.

Furthermore, according to the present invention, the shape memorized through the shape-memory treatment is not limited to a convex shape and the like, and can be freely set to, for example, a nested shape of a plurality of similar polygonal shapes (cobweb shape). With this, the measurement units can be arranged in a variety of manners. As described above, the measurement units can be arranged spreading two dimensionally or three dimensionally using the shape-memory unit according to the present invention, and the position of each measurement unit can be easily adjusted.

Further, according to the second aspect of the present invention, the shape change of the shape-memory unit can be further easily realized by applying stimulation to the shape-memory unit utilizing electrical heating.

Moreover, according to the third aspect of the present invention, it is possible to improve a spatial resolution as each measurement unit is a small electrode configured by separating the coating of the transmission wire and winding the wire around the holding unit. In addition, it is possible to make a cross section of the holding unit at a portion at which the measurement unit is provided to be substantially identical with that at a portion at which the measurement unit is not provided. Therefore, even if the shape-memory unit changes its shape within the body, possibility of damaging the dura mater or the arachnoid mater is reduced to a large extent.

Furthermore, according to the fourth aspect of the present invention, the shape memorized by shape-memory unit is the polygonal shape, and the holding unit holds the measurement units at the respective apices of the polygonal shape. When placing the measurement units within the body, it is possible to take an exact image of each measurement unit by intraoperative X-ray imaging, for example. However, it is not always possible to take an exact image of the measurement unit. Therefore, according to the fourth aspect of the present invention, if only part of information of sides of the polygonal shape can be taken even though an exact image of the measurement unit cannot be taken, it is possible to estimate a position of a measurement unit whose image has not been taken by obtaining information of the apices from the taken information. With this, it is further easily realized to suitably arrange the measurement units.

Moreover, according to the fifth aspect of the present invention, the positional estimation of the measurement units can be realized by a device using a computer, for example.

### BRIEF DESCRIPTION OF THE DRAWINGS

FIG. 1 is a view schematically illustrating a cross-section of a head 1 and a hole 19 for inserting a brain signal measurement device 21 as one example of the present invention.

FIG. 2(a) to FIG. 2(c) are views respectively schematically illustrating processing steps of inserting the brain signal measurement device 21 illustrated in FIG. 1 through the hole 19 and placing sensors 27₁-27₆.

FIG. 3 is a block diagram schematically illustrating a brain signal measurement system 31 as one example of the present invention.

FIG. 4(a) and FIG. 4(b) are views schematically illustrating a neighboring part 59 of sensors 33₄-33₆ illustrated in FIG. 3.

FIG. 5 is a graph showing experimental results of contact impedance between electrodes and normal saline solution.

FIG. 6 is a graph showing experimental results of electrical insulation between two electrodes.

### EMBODIMENTS OF THE INVENTION

Hereinafter, embodiments according to the present invention will be described with reference to the drawings. It should be noted that the present invention is not limited to these embodiments.

### EMBODIMENTS

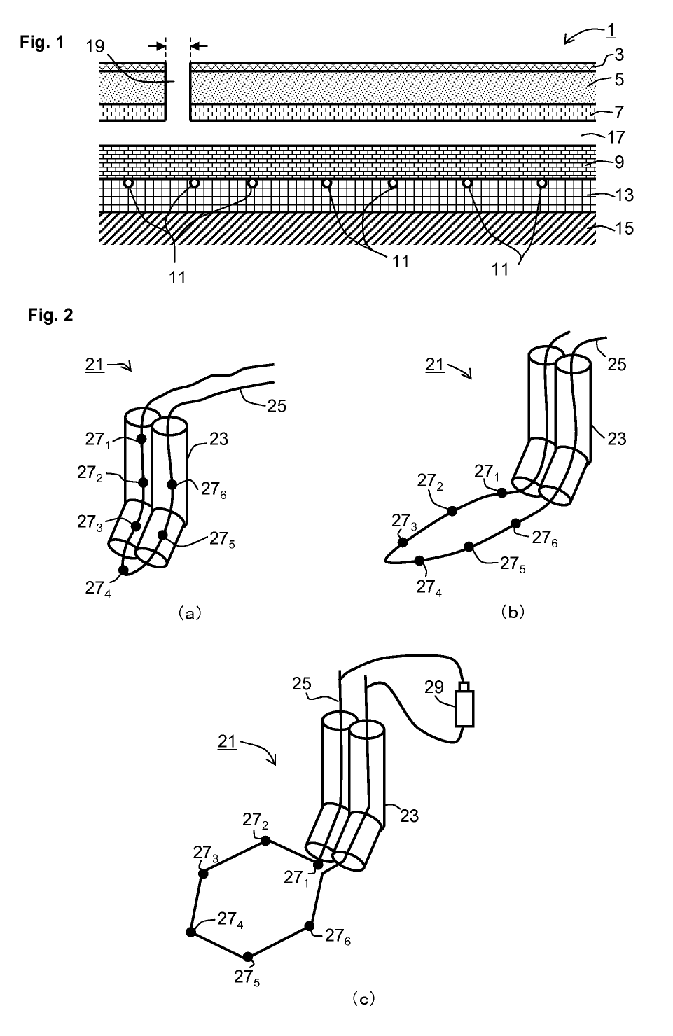

First, with reference to FIG. 1 and FIG. 2, an outline of processing steps of arranging six sensors 27₁-27₆ (hereinafter, when referring to the plural sensors, omitting suffixes and simply referred to as “the six sensors 27”, for example) of a brain signal measurement device 21 as one example of the present invention will be described.

As illustrated in FIG. 1, a head 1 has a hierarchical structure configured by a scalp and so on 3, a skull 5, a dura mater 7, an arachnoid mater 9, a blood vessel 11, a pia mater 13, and a brain 15 in the stated order from exterior. Although there is practically hardly any gap, it is still possible to arrange electrodes and the like within a space 17 between the dura mater 7 and the arachnoid mater 9. The brain signal measurement device according to the present invention is inserted into the space 17 between the dura mater 7 and the arachnoid mater 9 through a hole 19 penetrating through the scalp and so on 3, the skull 5, and the dura mater 7. The hole 19 can be realized as a hole having a diameter equal to or smaller than 1 cm, for example.

FIG. 2(a) to FIG. 2(c) are views schematically illustrating automatic expansion by electrical heating by the brain signal measurement device 21 according to the present invention (corresponding to a “brain signal measurement device” in Claims). The brain signal measurement device 21 includes introduction tubes 23 having a pair of tubes provided in parallel with one end of each of the pair of tube being configured as a peak having a diameter equal to or smaller than 1 cm and to be inserted into the head through the hole 19, and a holding unit 25 configured as a line (corresponding to a “holding unit” in Claims). The holding unit is passing through one of the introduction tubes from one end not to be inserted into the head and exiting from the other end to be inserted into the head, and then inserted into the other of the introduction tubes from one end to be inserted into the head and exiting from the other end not to be inserted into the head. The holding unit 25 holds the six sensors 27 (corresponding to “measurement units” in Claims). The holding unit 25 includes an SMA guide made of a shape-memory alloy (corresponding to “shape-memory unit” in Claims), and a heat shrinkable tube as a thermal and electrical insulator that covers the SMA guide (corresponding to “stimulation blocking unit” in Claims). Examples of a material of the SMA guide include a titanium-nickel alloy. This alloy has been proved to be biocompatible, and variously utilized in medical applications including applications for biological implantation. Other examples may include an iron-based shape-memory alloy. Further, examples of a material of the sensors 27 include platinum (platinum) and a platinum iridium alloy. These alloys have also been proved to be biocompatible, and variously utilized in medical applications including applications for electrodes for catheters and pacemakers. In FIG. 4, platinum is taken as an example. Further, examples of a material of the heat shrinkable tube include a fluorine resin, PTFE (for example, Teflon (registered trademark)). Likewise, there is a fluorine resin whose biocompatibility has been proved.

As for this embodiment, the SMA guide is more flexible than the dura mater 7 and the arachnoid mater 9 at least in temperature from room temperature to body temperature, and takes a shape that has been memorized through a shape-memory treatment when the temperature rises up to or above a predetermined temperature by electrical heating. Here, a central portion of the SMA guide is subjected to the shape-memory treatment to memorize a regular hexagon, and the sensors 27 are held by the holding unit 25 respectively at positions corresponding to apices of the regular hexagon of the SMA guide.

Referring to FIG. 2(a), when the introduction tubes 23 are inserted into the hole 19, the holding unit 25 is not exposed from the ends of the introduction tubes 23 to be inserted into the head, and the introduction tubes 23 are inserted into the hole 19 while the sensors 27 are contained within the introduction tubes 23.

Next, referring to FIG. 2(b), after the insertion of the introduction tubes 23 into the hole 19, when the ends of the introduction tubes 23 reach the space 17 between the dura mater 7 and the arachnoid mater 9, the introduction tubes 23 are fixed to the hole 19, and a central portion of the holding unit 25 (at least a part at which the regular hexagon is memorized through the shape-memory treatment) is inserted into the space 17 between the dura mater 7 and the arachnoid mater 9 from the ends of the introduction tubes 23 inserted into the head.

Then, referring to FIG. 2(c), by electrically heating the SMA guide of the holding unit 25 by a power source 29 (corresponding to a “stimulation device” in Claims), the SMA guide of the holding unit 25 changes its shape into the regular hexagon that has been memorized through the shape-memory treatment. The sensors 27 respectively held at the apices of the regular hexagon move to predetermined positions along with a shape change of the SMA guide. The diameter equal to or smaller than 1 cm is sufficient for the hole 19, and it is possible to place the subdural electrodes with a minimally invasive operation, because the electrodes can be expanded such that a ring is expanded after being inserted into the subdural space through such a hole.

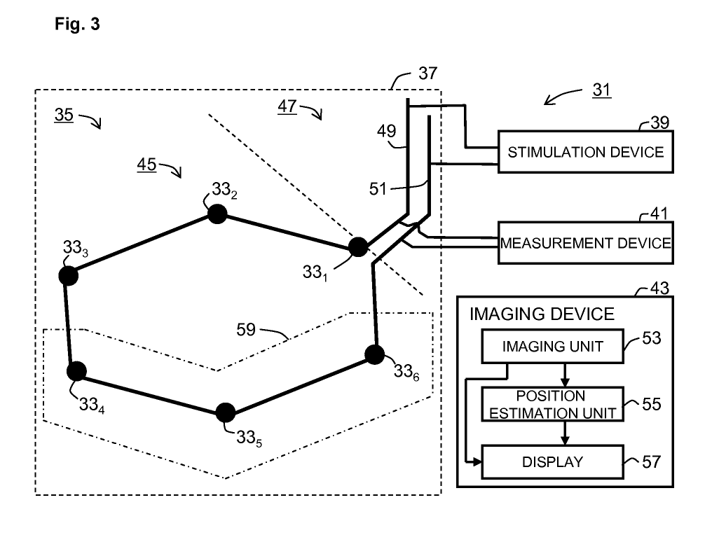

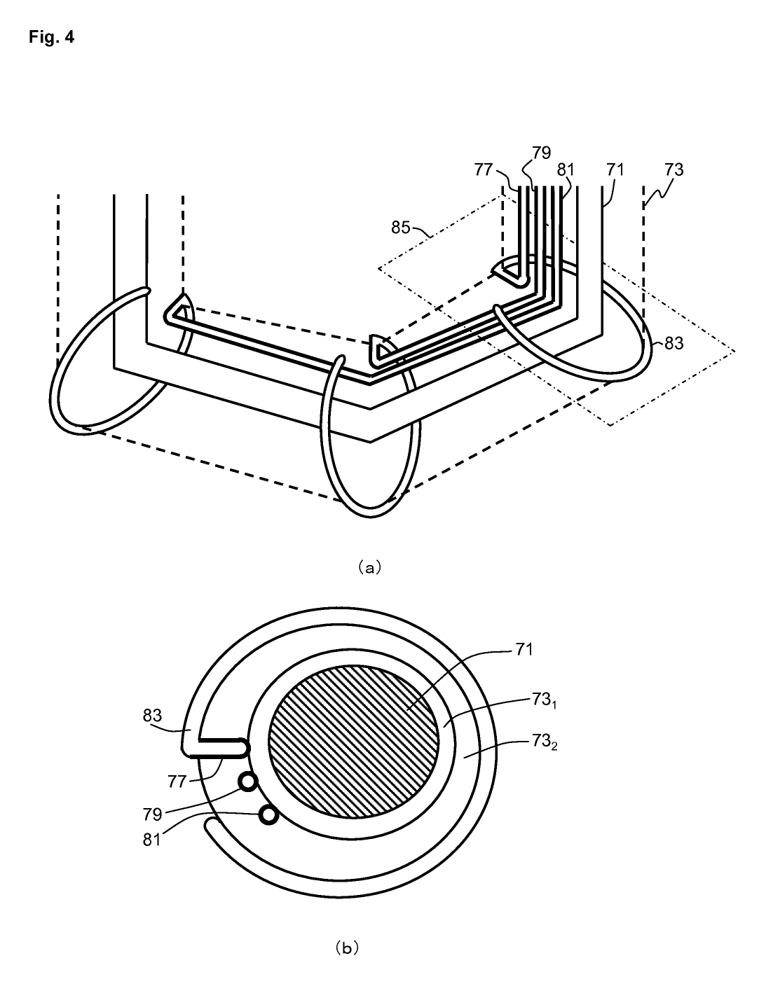

Next, referring to FIG. 3, a configuration of a brain signal measurement system 31 as one example of the present invention (corresponding to the “brain signal measurement system” in Claims) will be specifically described.

The brain signal measurement system 31 includes a brain signal measurement device 37 having a plurality of sensors 33 to be provided in a space between a dura mater and a arachnoid mater, and a holding unit 35 configured to hold the plurality of sensors 33. Each of the sensors 33 is configured to measure a brain signal. The brain signal measurement system 31 further includes a stimulation device 39 configured to supply stimulation to the SMA guide of the holding unit 35, a measurement device 41 configured to receive measurement results from the sensors 33, and an imaging device 43 configured to exogenously take an image using X-ray and the like and display the image.

The holding unit 35 includes a sensor holding unit 45 having a shape-memory property of a regular hexagonal shape memorized through a shape-memory treatment, and a stimulus introduction unit 47 configured to introduce stimulation from the stimulation device 39 to the sensor holding unit 45.

In this embodiment, the sensors 33 are positioned at each of the apices of the hexagon, and the sensor holding unit 45 and the stimulus introduction unit 47 are configured such that a shape-memory alloy (SMA guide) that exhibits the shape-memory property by heating (the SMA guide of the sensor holding unit 45 corresponds to the “shape-memory unit ” in Claims) is covered by a heat shrinkable tube as a thermal and electrical insulator (corresponding to the “stimulation blocking unit” in Claims).

The stimulation device 39 changes positions of the sensors 33 by heating the sensor holding unit 45 inserted in the body (see FIG. 2 (c)) by conducting electricity from one edge 49 to the other edge 51 of the holding unit 35 to change the shape of the sensor holding unit 45 based on the shape-memory property of the sensor holding unit 45 into the shape memorized in advance through the shape-memory treatment. The electrical heating is instantaneous heating by a current of about 1-2 A, and the heat is not conducted outside as thermal conductivity of the insulator is low. This makes it easy to place the sensors 33. Further, as the sensor holding unit 45 becomes flexible in temperature from room temperature to body temperature, positional adjustment within the head and removal from the head are also facilitated. Therefore, the insertion and the removal of the subdural electrodes can be done more easily.

The measurement device 41 obtains measurement results of the sensors 33₁, 33₂, and 33₃ closer to an edge 49 of the holding unit 35 through the edge 49, and obtains measurement results of the sensors 33₄, 33₅, and 33₆ closer to the other edge 51 of the holding unit 35 through the edge 51. A configuration of the holding unit 35 in a periphery 59 of the sensors 33₄, 33₅, and 33₆ will be specifically described later with reference to FIG. 4.

The imaging device 43 includes an imaging unit 53 (corresponding to an “imaging unit” in Claims), a position estimation unit 55 (corresponding to a “position estimation unit ” in Claims) and a display 57. The imaging unit 53 is capable of exogenously taking an image such as an intraoperative X-ray image of the SMA guide of the sensor holding unit 45 within the head. The position estimation unit 55 is configured to perform image processing to information of the image taken by the imaging unit 53, estimate a position of a directly detectable one of the sensors 33 based on a detected position, and estimate a position of an directly un-detectable one of the sensors 33 by estimating a position of an apex based on information of sides of the polygonal shape of the sensor holding unit 45. The display 57 displays imaging results by the imaging unit 53 and position estimation results by the position estimation unit 55.

Next, referring to FIG. 4, the configuration of the holding unit 35 in the periphery 59 of the sensors 33₄, 33₅, and 33₆ will be specifically described.

The holding unit 35 includes an SMA guide 71 made of a shape-memory alloy, and a heat shrinkable tube 73 which is a thermal and electrical insulator covering the SMA guide 71. In FIG. 4 (a), the heat shrinkable tube 73 is represented by a dashed line in order to clearly illustrate an internal structure.

Coated platinum wires 77, 79, and 81 are coated by Teflon (registered trademark), and respectively for providing the sensors 33₆, 33₅, and 33₄. The coated platinum wires 77, 79, and 81 are partially covered by the heat shrinkable tube 73. One end of the coated platinum wire 77 is electrically connected to a measurement device 41 in a coated state. The other end of the coated platinum wire 77 is pulled out of the heat shrinkable tube 73 at a portion corresponding to an apex of the hexagon to be provided as a bare platinum wire 83, which is wound around the heat shrinkable tube 73 to be used as an electrode that functions as the sensor 33₆. By miniaturizing electrodes in this manner, it is possible to improve a spatial resolution. Likewise, the coated platinum wires 79 and 81 are pulled out of the heat shrinkable tube 73 at respective portions corresponding to apices of the hexagon to be provided as bare platinum wires, which are wound around the heat shrinkable tube 73 to be used as electrodes that respectively function as the sensors 33₅ and 33₄.

FIG. 4(b) is a cross-sectional view at a cross section 85 in FIG. 4(a). Here, the heat shrinkable tube 73 has a hierarchical structure configured by a heat shrinkable tube 73₁ covering the SMA guide 71, and a heat shrinkable tube 73₂ disposed outside and covering the heat shrinkable tube 73₁ as well as the coated platinum wires 77, 79, and 81.

The following describes a specific example of the brain signal measurement device 37. A wire diameter of the holding unit 35 is 0.3 mm. A material of the SMA guide 71 is a Ni—Ti alloy of 54.9 wt % (wt %: percent concentration by mass), and its DC resistance value is about 23 Ω. The sensor holding unit 45 is subjected to the shape-memory treatment to memorize a regular hexagon 2 cm on each side, and the stimulus introduction unit 47 is 4 cm. The shape-memory treatment is performed by heating for 40 minutes at 370 degrees Celsius. The heat shrinkable tube is made of fluoropolymer, and its film thickness after contraction is 0.25 mm.

Under the conditions of the material, the shape, and the shape-memory treatment described above, and in air at 27 degrees Celsius, the memorized shape is restored by applying a current of 0.2 m A to the SMA guide for about two seconds. At the same time, brain cells may not be damaged due to heat as the heat is hardly transmitted outside the heat shrinkable tube by heating for two seconds.

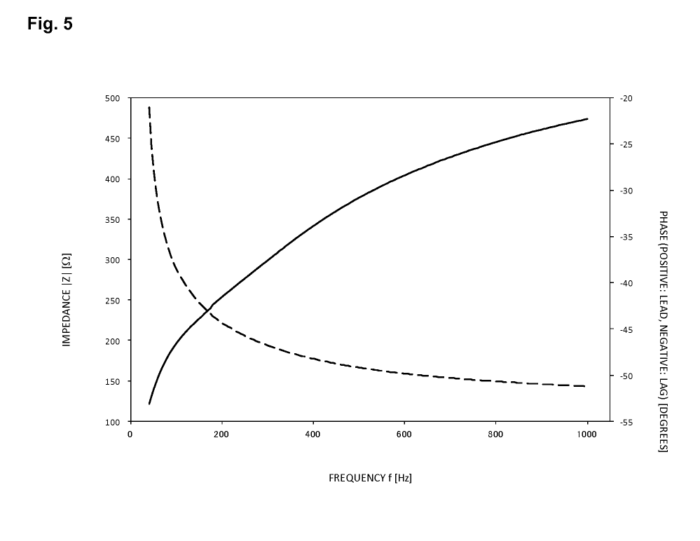

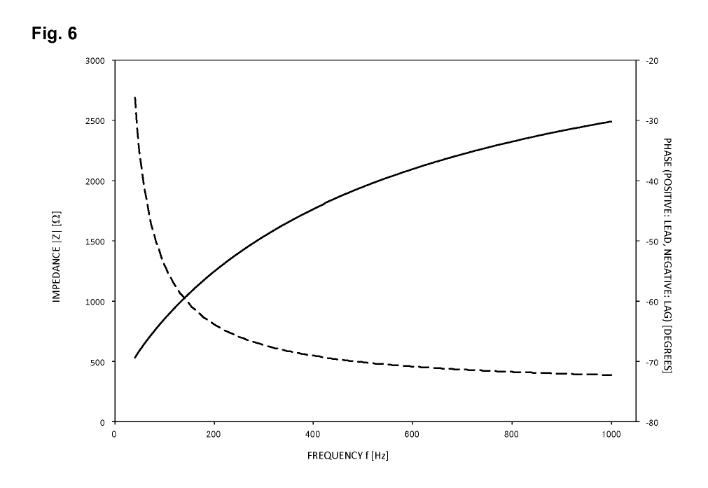

FIG. 5 is a graph showing experimental results of contact impedance between the electrodes and normal saline solution. FIG. 6 is a graph showing experimental results of electrical insulation between two electrodes. In FIG. 5 and FIG. 6, the horizontal axis indicates frequency, and the vertical axis indicates impedance (dashed line) and phase lag (solid line).

The experiment is conducted in normal saline solution that comprehensively simulates spinal fluid, nerve cells, and the like. A space between electrodes is filled with normal saline solution, and the electrodes are electrically connected. In the following, a case in which a certain brain wave is detected by two adjacent sensors is described taking the sensor 33₄ and the sensor 33₅ illustrated in FIG. 3 as an example.

A brain wave generated immediately under the sensor 33₄ is transmitted to the electrodes from a nerve cell as a signal source through “contact impedance between the electrodes and normal saline solution” (see FIG. 5). At the same time, the brain wave is also transmitted through a route “from the signal source to the sensor 33₅ through the spinal fluid”. However, the signal that has been transmitted through the latter route attenuates depending on, for example, a distance between the signal source and the electrode. The “impedance between two electrodes” shows a degree of the attenuation (see FIG. 6).

In this experiment, in a signal frequency band of the brain wave, the impedance between the electrodes is about 10 times higher than the contact impedance between the electrodes and normal saline solution. Therefore, it is possible to estimate that a brain wave immediately under one sensor attenuates down to one tenth at an adjacent electrode. As described above, the contact impedance between electrodes and normal saline solution is sufficiently small in the signal frequency band of a brain wave, and the electrical insulation between two electrodes is sufficiently high. Accordingly, it is estimated that there is no possibility that amplitude of measured brain wave is too small and buried in Noise and that a cross talk between brain waves measured by different electrodes may occur.

It should be noted that the shape-memory unit is not limited to the shape-memory alloy, and may be, for example, a shape-memory resin that exhibits a shape-memory property. In this case, stimulation to be applied is suitably selected according to the material. In addition, the shape to be memorized is not limited to a regular hexagon, and may be freely set to, for example, a nested shape of a plurality of similar polygonal shapes (cobweb shape).

Further, it is possible that the imaging device 43 is provided with an input unit through which a user inputs positional information of the sensors 33, the position estimation unit 55 compares the inputted positional information of the sensors 33 with estimated positional information, and the display 57 displays comparison results.

Moreover, the present invention can be considered as a measurement device including a coated and columnar transmission wire and a tubular insulator covering the transmission wire, one end of the transmission wire being pulled out of the insulator to be provided as an electrode with its coating separated and being wound around the insulator at the same cross-section, and the other end of the transmission wire outputting a signal measured by the electrode while blocking an influence given to and from exterior of the insulator.

### DESCRIPTION OF REFERENCE SIGNS

21: Brain Signal Measurement Device

23: Introduction Tube

25: Holding Unit

27₁, . . . 27₆: Sensor

29: Power Source

31: Brain Signal Measurement System

33₁, . . . 33₆: Sensor

35: Holding Unit

37: Brain Signal Measurement Device

39: Stimulation Device

43: Imaging Device

45: Sensor Holding Unit

47: Stimulus Introduction Unit

53: Imaging Unit

55: Position Estimation Unit

1-11. (canceled)

12. A brain signal measurement system, comprising:
a brain signal measurement device including:
a plurality of measurement units provided in a space between a dura mater and an arachnoid mater, and each configured to measure a brain signal; and
a holding unit configured to hold the plurality of measurement units, wherein
the holding unit includes:
a shape-memory unit having a shape-memory property exerted by predetermined stimulation, and configured to change positions of the plurality of measurement units by a shape change of the shape-memory unit based on the shape-memory property;
a stimulus introduction unit configured to supply stimulation to the shape-memory unit from exterior; and
a stimulation blocking unit covering the shape-memory unit, and configured to block an influence given to exterior by a predetermined phenomenon other than the shape change of the shape-memory unit by supplying the predetermined stimulation to the shape-memory unit from exterior,
the holding unit in a non-stimulated state is more flexible than the dura mater and the arachnoid mater, and
the shape-memory unit changes the shape thereof into a shape previously memorized through a shape-memory treatment and positions the plurality of measurement units, the change of the shape being based on the shape-memory property, the change of the shape being caused by providing the holding unit between the dura mater and the arachnoid mater and by supplying the predetermined stimulation using a stimulation device via the stimulus introduction unit.

13. The brain signal measurement system according to claim 12, wherein
the shape-memory unit is configured by a shape-memory alloy, and changes the shape thereof based on the shape-memory property by being heated up to or above a predetermined temperature,
the stimulation supplied by the stimulation device is electrical heating, and
the stimulation blocking unit is a thermal and electrical insulator.

14. The brain signal measurement system according to claim 12, wherein
the brain signal measurement device includes introduction tubes having a pair of tubes provided in parallel with one end of each of the pair of tubes to be inserted between the dura mater and the arachnoid mater,
the holding unit is configured as a line, passing through one of the introduction tubes from one end not to be inserted between the dura mater and the arachnoid mater and out of the other end to be inserted between the dura mater and the arachnoid mater, and passing through the other of the introduction tubes from one end to be inserted between the dura mater and the arachnoid mater and out of the other end not to be inserted between the dura mater and the arachnoid mater.

15. The brain signal measurement system according to claim 12, wherein
each of the measurement units is configured by a transmission wire electrically connected to a measurement device and capable of transmitting a measurement result obtained at an electrode,
a portion of the transmission wire other than the electrode is coated, and at least a part of the portion of the transmission wire other than the electrode is covered by the stimulation blocking unit in addition to the coating so as to allow transmission of the measurement result obtained at the electrode while blocking an influence given to and from exterior, and
the electrode is configured by the transmission wire with its coating separated and is wound around the holding unit.

16. The brain signal measurement system according to claim 12, wherein
the shape-memory unit is shape-memory treated to memorize a polygonal shape having apices of a number of or greater than a number of the plurality of measurement units,
the measurement units are respectively held at the apices of the polygonal shape of the holding unit.

17. The brain signal measurement system according to claim 16, further comprising:
an imaging device including:
an imaging unit capable of exogenously taking an image of the shape-memory unit; and
a position estimation unit configured to:
perform image processing to information of the image taken by the imaging unit;
estimate a position of a detected one of the measurement units based on a detected position; and
estimate a position of an un-detected one of the measurement units to be a position of an apex estimated based on information of sides of the polygonal shape of the shape-memory unit.

18. A measurement system comprising an in vivo measurement device, the in vivo measurement device comprising:
a plurality of measurement units provided in a predetermined in vivo space, and each configured to measure a signal; and
a holding unit configured to hold the plurality of measurement units, wherein
the in vivo measurement device includes introduction tubes having a pair of tubes provided in parallel with one end of each of the pair of tubes to be inserted into the predetermined in vivo space,
the holding unit includes:
a shape-memory unit having a shape-memory property exerted by predetermined stimulation, and configured to change positions of the plurality of measurement units by a shape change of the shape-memory unit based on the shape-memory property;
a stimulus introduction unit configured to supply stimulation to the shape-memory unit from exterior; and
a stimulation blocking unit covering the shape-memory unit, and configured to block an influence given to exterior by a predetermined phenomenon other than the shape change of the shape-memory unit by supplying the predetermined stimulation to the shape-memory unit from exterior,
the holding unit in a non-stimulated state is more flexible than an outer wall of the predetermined in vivo space,
the shape-memory unit changes the shape thereof into a shape previously memorized through a shape-memory treatment and positions the plurality of measurement units, the change of the shape being based on the shape-memory property, the change of the shape being caused by providing the holding unit in the predetermined in vivo space and by supplying the predetermined stimulation using a stimulation device via the stimulus introduction unit,
the holding unit is configured as a line, passing through one of the introduction tubes from one end not to be inserted in the predetermined in vivo space and out of the other end to be inserted in the predetermined in vivo space, and passing through the other of the introduction tubes from one end to be inserted in the predetermined in vivo space and out of the other end not to be inserted in the predetermined in vivo space.

19. The measurement system according to claim 18, wherein
the shape-memory unit is configured by a shape-memory alloy, and changes the shape thereof based on the shape-memory property by being heated up to or above a predetermined temperature,
the stimulation supplied by the stimulation device is electrical heating,
the stimulation blocking unit is a thermal and electrical insulator, and
the stimulation device supplies the stimulation by conducting electricity from one end to the other end of the holding unit.

20. The measurement system according to claim 18, wherein
each of the measurement units is configured by a transmission wire electrically connected to a measurement device and capable of transmitting a measurement result obtained at an electrode,
a portion of the transmission wire other than the electrode is coated, and at least a part of the portion of the transmission wire other than the electrode is covered by the stimulation blocking unit in addition to the coating so as to allow transmission of the measurement result obtained at the electrode while blocking an influence given to and from exterior, and
the electrode is configured by the transmission wire with its coating separated and is wound around the holding unit.

21. The measurement system according to claim 18, wherein
the shape-memory unit is shape-memory treated to memorize a polygonal shape having apices of a number of or greater than a number of the plurality of measurement units,
the measurement units are respectively held at the apices of the polygonal shape of the holding unit.

22. The measurement system according to claim 21, further comprising:
an imaging device including:
an imaging unit capable of exogenously taking an image of the shape-memory unit; and
a position estimation unit configured to:
perform image processing to information of the image taken by the imaging unit;
estimate a position of a detected one of the measurement units based on a detected position; and
estimate a position of an un-detected one of the measurement units to be a position of an apex estimated based on information of sides of the polygonal shape of the shape-memory unit.
